(19) United States
(12) Patent Application Publication
(10) Pub. No.: US 2006/0089712 A1
(43) Pub. Date: Apr. 27, 2006

(54) Accommodative intraocular lens

(76) Inventor: Francois Malecaze, Toulouse (FR)

Correspondence Address: YOUNG & THOMPSON, 745 SOUTH 23RD STREET, 2ND FLOOR, ARLINGTON, VA, 22202, US

(21) Appl. No.: 10/520,072

(22) PCT Filed: Jun. 30, 2003
(86) PCT No.: PCT/FR03/02021

§ 371 Date: Dec. 9, 2005

(30) Foreign Application Priority Data
Jul. 3, 2002 (FR) 02/08338

(51) Int. Cl. A61F 2/16

(52) U.S. Cl. 623/6.11

(57) ABSTRACT

The invention concerns an accommodative intraocular lens for capsular bag comprising a central optical part and a peripheral haptic part, the optic part having an forward position for accommodation and a rest position for far vision. The invention is characterized in that the haptic part (20) comprises a radially extending zone for displacing the optic part (10) towards the forward position. The radially extending zone (21) may include a gusset (22) with at least one crimp and/or made of a more flexible material than the remainder of the haptic part.

The present invention relates to intraocular lenses, also known as intraocular implants, designed to replace crystalline lenses affected by cataracts, after ablation thereof, and more particularly to accommodative intraocular lenses.

The intact crystalline lens enables a person to see close up or far away thanks to the mechanism known as accommodation. Accommodation is linked to the variation in the shape of the crystalline lens by contraction of the ciliary muscle. This mechanism is still not fully understood.

According to the most widely accepted theory, put forward by Helmholtz, during accommodation, the contraction of the ciliary muscle leads to relaxation of the zonular fibers attached to the equator of the capsular sac of the crystalline lens. This relaxation allows the crystalline lens to “bulge”, the radii of curvature of the anterior and posterior faces decreasing, thereby increasing the power or vergence of the crystalline lens. Similarly, during accommodation, the anterior face of the crystalline lens moves forward, towards the cornea, because of vitreous thrust induced by an increase in pressure.

There are other theories of the accommodation mechanism. According to that of Schachar, which contradicts that of Helmholtz, the contraction of the ciliary muscle tensions the zonule, which is said to apply traction at the equator and to be responsible for the deformation of the central part of the crystalline lens.

Similarly, the role of the vitreous in accommodation is controversial. According to some, the vitreous opposes modification of the shape of the posterior face of the crystalline lens during accommodation but contributes to forward movement of the crystalline lens in the direction of the cornea.

Moreover, presbyopia reduces the accommodation capacity of the natural crystalline lens, and mutually consistent studies have shown that the contraction of the ciliary muscle is at least partially preserved when a person suffers from presbyopia.

The ablation of the crystalline lens is usually effected by capsulotomy of the anterior capsule or leaf of the capsular sac, followed by phacoemulsification of the crystalline lens and cleaning of the site. Thereafter, the implant is introduced into the interior of what remains of the capsular sac, namely the posterior capsule and the remaining annular peripheral portion of the anterior capsule. The natural kinetics of accommodation are affected by the capsulotomy, the extraction of the crystalline lens and, to a lesser degree, the implanting of an intraocular lens.

However, accommodative intraocular lenses have been designed to exploit remaining forces in a pseudophakic eye, i.e. after extraction of the crystalline lens and implantation of an intraocular lens. These accommodative intraocular lenses have not given full satisfaction, in particular because there is insufficient displacement in the posterior-anterior direction with the new kinetics of the capsular sac of a pseudophakic eye.

The document WO 97/43984 describes an intraocular lens having an elastically deformable intermediate region for modifying the angle of inclination of this zone relative to a plane normal to the optical axis of the lens and therefore having insufficient accommodation. The same applies to the document WO 01/60286 in which an intraocular lens is hinged to a shoe.

The present invention aims to overcome the drawbacks referred to above. It consists in a novel accommodative intraocular lens better able to exploit the new kinetics of the capsular sac of a pseudophakic eye and in particular the vitreous hyperpressure. The contraction of the ciliary muscle that is at the root of the accommodation mechanism induces an increase in the vitreous pressure. The vitreous is surrounded by the sclerotic, which is substantially undeformable, and by the posterior capsule, which is deformed as a result of the increase in the vitreous pressure. According to Dr Coleman (“On the hydraulic suspension theory of accommodation” Tr. Am. Opht. Soc. Vol. 84, 1986), the variation of vitreous pressure in primates during accommodation is from 2 to 10 cm of water, i.e. from approximately 200 Pa to approximately 1000 Pa. Pressure variations of this magnitude would cause a displacement in the posterior-anterior direction from approximately 0.5 mm to approximately 2 mm, i.e. sufficient movement for good accommodation by an intraocular lens.

The new kinetics also involve displacement of the apex of the ciliary muscle and of the equator of the crystalline sac, both radially towards the optical axis of the eye and in the anterior direction. One object of the present invention is to exploit this conjoint and linear displacement of the apex of the ciliary muscle and the equator of the crystalline sac to induce accommodation of an intraocular lens.

The present invention provides an accommodative intraocular lens for implantation in the capsular sac comprising a central optical part and a peripheral haptic part, the optical part having a forward position for accommodation and a rest position for far vision, characterized in that the haptic part comprises a radial expansion zone for displacing the optical part towards the forward position.

In practice this zone is situated between the peripheral edge portion of the optical part and that of the haptic part. It may extend over the whole or a portion of the radial distance between the peripheral edge portion of the optical part and the peripheral edge portion of the haptic part. Its circumference is preferably the same as the circumference of the haptic part inside which it is situated.

The expansion potential of the radial expansion area as determined between a point on the periphery of the optical part and a point on the same radius on the periphery of the haptic part is from 0.2 mm to 1.6 mm. This expansion potential of the radial expansion area allows axial displacement of the optical part of from 0.8 mm to 2.0 mm to provide good accommodation of near vision. The elasticity of the radial expansion zone in the forward accommodation position returns the optical part to the rest position for far vision. In a preferred embodiment of the invention, the radial expansion zone comprises a bellows. In other words, this radial expansion zone comprises at least one undulation and is substantially annular or circumferential, possibly being interrupted by a plurality of radial notches opening onto the periphery of the haptic part, to encourage posterior-anterior displacement, or interrupted by gaps between radial arms constituting haptic members extending between the peripheral edge portion of the optical part and that of the haptic part.

In one preferred embodiment, the bellows comprises at least two undulations, one opening in the anterior direction and the other in the posterior direction, the one opening in the anterior direction preferably being disposed at the periphery of the optical part.

In one embodiment of the invention, the peripheral edge portion of the haptic part has posterior and anterior right angles.

In a different embodiment, the haptic part comprises a peripheral gutter to provide separation parallel to the optical axis between the remainder of the anterior capsule and the posterior capsule of a pseudophakic eye.

In another embodiment, the radial expansion zone is made from a less rigid material, and thereby constitutes a more flexible zone, with the result that expansion results from stretching of this more elastic material. Moreover, the bellows may be made at least in part from a material of higher elasticity, so that expansion results both from flattening of the undulations or bellows and stretching of the part made from a material having a higher elasticity.

In one preferred embodiment, the haptic part comprises at least two haptic members, each with a radial expansion zone comprising a bellows or one or more undulations and/or made from a material having a higher elasticity. These haptic members preferably have a circumference at their periphery that is greater than their circumference at the junction with the optical part.

Features and advantages of the invention will emerge from the following description, which is given by way of example and with reference to the appended drawings:

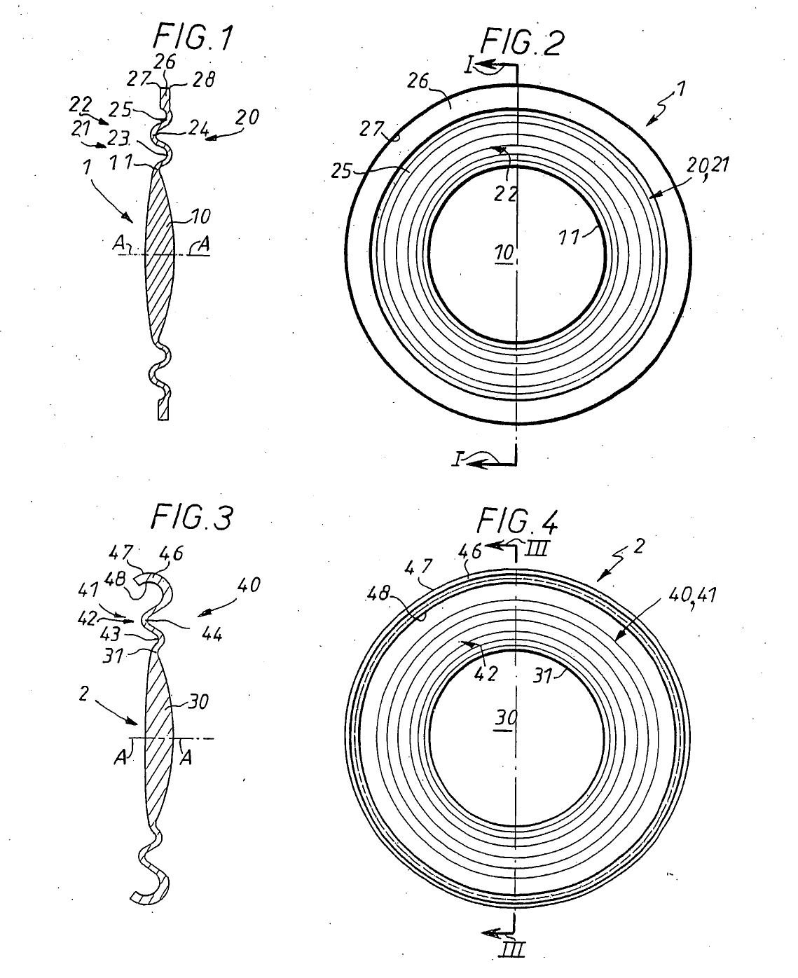

FIG. 1 is a view of a first embodiment of an accommodative intraocular lens of the present invention in section taken along the line I-I in FIG. 2;

FIG. 2 is a front view of the FIG. 1 intraocular lens;

FIG. 3 is a view of a second embodiment in section taken along the line III-III in FIG. 4;

FIG. 4 is a front view of the FIG. 3 intraocular lens;

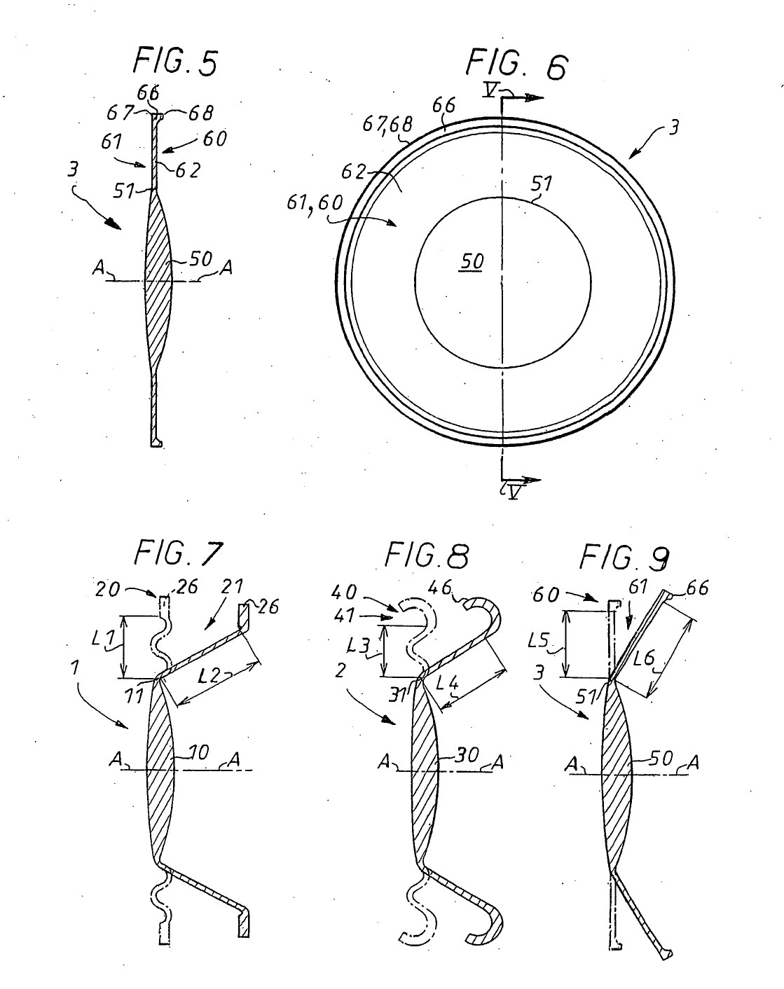

FIG. 5 is a view of a third embodiment in section taken along the line V-V in FIG. 6;

FIG. 6 is a front view of the FIG. 5 intraocular lens;

FIG. 7 is a view in section of the accommodative intraocular lens from FIGS. 1 and 2 showing the expansion of the radial expansion zone of the haptic part (shown in continuous line) relative to the rest configuration (shown in chain-dotted line);

FIG. 8 is a view in section of the accommodative intraocular lens from FIGS. 3 and 4 showing the expansion of the radial expansion zone of the haptic part (shown in continuous line) relative to the rest configuration (shown in chain-dotted line);

FIG. 9 is a view in section of the accommodative intraocular lens from FIGS. 5 and 6 showing the expansion of the radial expansion zone of the haptic part (shown in continuous line) relative to the rest configuration (shown in chain-dotted line);

FIGS. 10 and 11 show the FIG. 1 intraocular lens implanted in the eye and respectively in the rest position and the accommodation position;

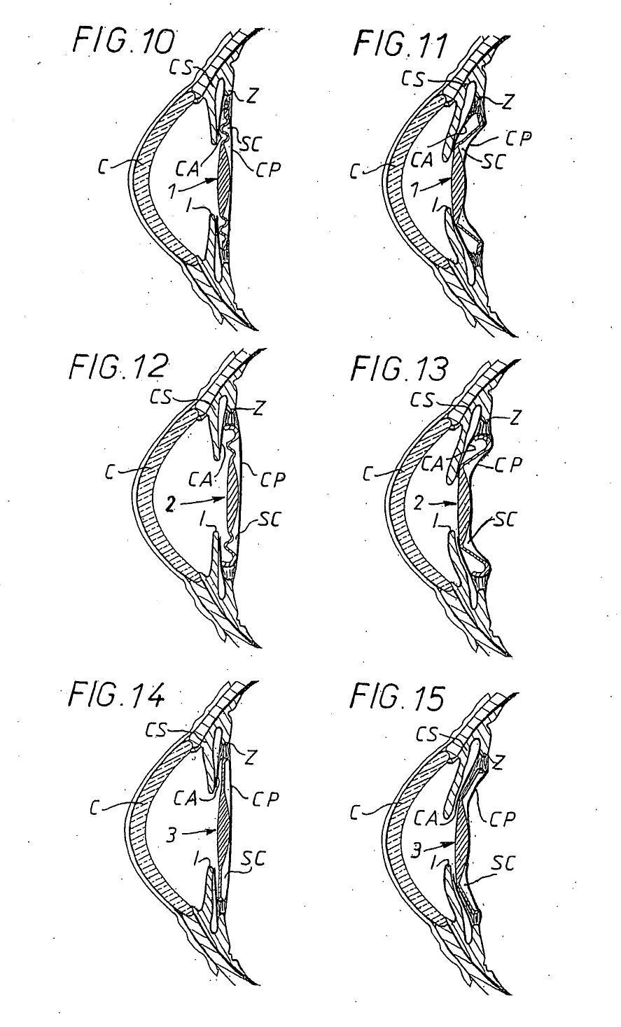

FIGS. 12 and 13 show the FIG. 3 intraocular lens implanted in the eye and respectively in the rest position and the accommodation position;

FIGS. 14 and 15 show the FIG. 5 intraocular lens implanted in the eye and respectively in the rest position and the accommodation position;

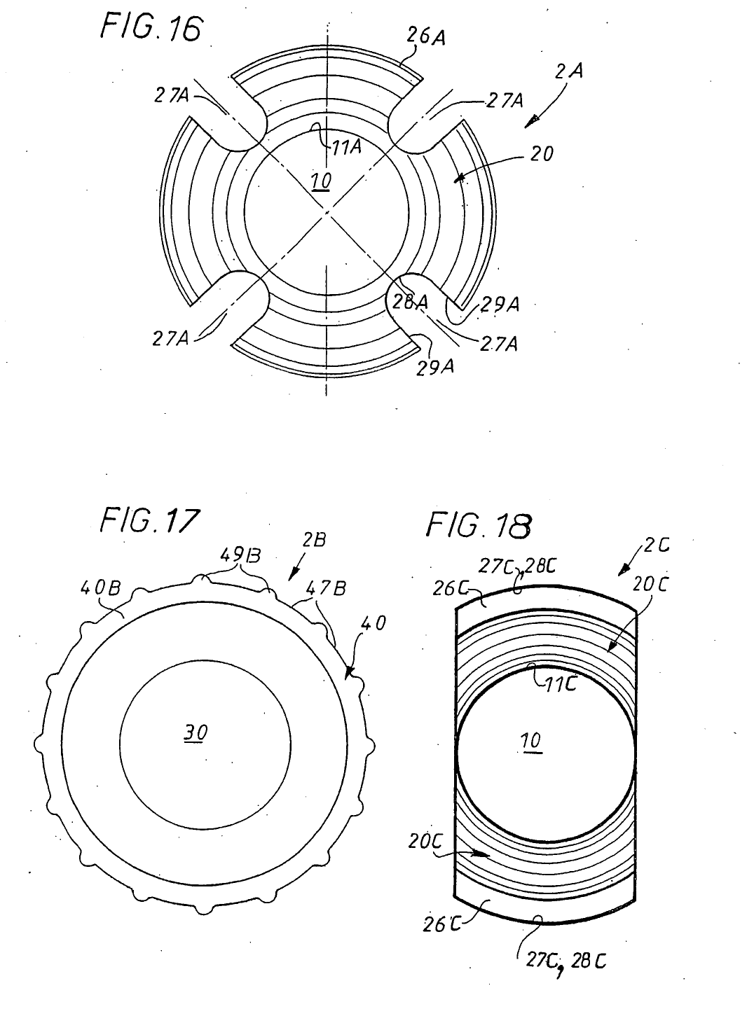

FIG. 16 is a front view analogous to FIG. 2 of a variant of the first embodiment in which the haptic part comprises a plurality of radial notches;

FIG. 17 is a view analogous to FIG. 2 of a second variant of the first embodiment in which the haptic part comprises a plurality of bosses along the circumference adapted to face the equator of the capsular sac;

FIG. 18 is a front view analogous to FIG. 6 of a variant of the third embodiment in which the haptic part comprises two haptic bellows members;

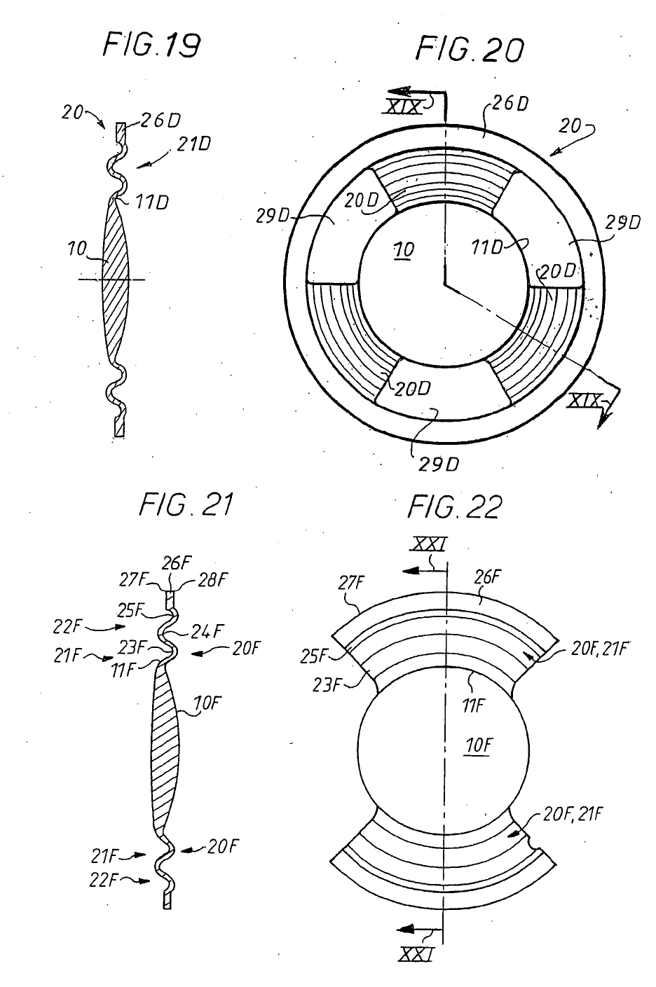

FIG. 19 is a view analogous to that of FIG. 1 of another variant of the accommodative intraocular lens;

FIG. 20 is a front view of the FIG. 19 intraocular lens;

FIG. 21 is a view of a preferred embodiment of an accommodative intraocular lens in section taken along the line XXI-XXI in FIG. 22; and

FIG. 22 is a front view of the FIG. 21 intraocular lens.

In the embodiment shown in FIGS. 1 and 2, the accommodative intraocular lens 1 comprises a central optical part 10 having an optical axis A-A and a peripheral haptic part 20 extending circumferentially around the optical part. The intraocular lens is preferably made entirely or partly from flexible material, such as a hydrophilic acrylic or polyHEMA. However, any other flexible material used for the production of intraocular lenses may be used. As shown here, the optical part 10 is biconvex. It may have other shapes, in particular plano-convex, or even concave-convex. The posterior face of the optical part is preferably convex and conformed to espouse the central region of the posterior capsule and thereby assure good transmission of vitreous hyperpressure.

The peripheral edge portion of the optical part may be provided with a sharp annular edge projecting to the rear to reduce migration of epithelial cells between the optical part and the posterior capsule.

According to the invention, the haptic part 20 comprises a radial expansion zone 21. In this first embodiment, the radial expansion zone 21 comprises a bellows 22 or one or more undulations, of which the first undulation 23 is open on the anterior side and is situated in the immediate vicinity of the periphery 11 of the optical part 10. This first annular undulation 23 is surrounded by a second annular undulation 24 that is open on the posterior side and surrounded by a third annular undulation 25 that is open on the anterior side. In this embodiment, the first two undulations have substantially the same configuration, although extending in opposite directions, while the third undulation 25 is narrower in the radial direction than the other two undulations. In this embodiment, the bellows 22 has a substantially sinusoidal shape extending from the periphery 11 of the optical part to the peripheral edge portion 26. In a variant that is not shown, the bellows may have a somewhat more sawtooth shape.

In another variant that is not shown, the third undulation is replaced at least in part by a substantially plane annular zone continuous with the peripheral edge portion 26, which is preferably annular and continuous, and has a substantially rectangular section, with a radial dimension, for example 0.6 mm, greater than its axial dimension, for example 0.3 mm. The external edge of the peripheral edge portion 26 has an anterior sharp or square corner edge 27 and a posterior sharp or square corner edge 28. The radial expansion zone 21 forming the bellows 22 or comprising one or more undulations preferably has a substantially constant thickness from the periphery of the optical part 11 to the peripheral edge portion 26. The depth of the first two undulations is the same and from approximately 0.40 mm to approximately 0.70 mm and the subtended angle is from approximately 50′ to approximately 70°.

In a second preferred embodiment, shown in FIGS. 21 and 22, the haptic part 20 comprises two haptic members 20F extending in opposite directions from the peripheral edge portion 11F of the optical part 10F. Each of these haptic members 20F has substantially the same radial section as the haptic part 20 of the embodiment shown in FIGS. 1 and 2. The corresponding parts are designated by the same reference numbers with the suffix F. The circumference of each haptic member 20F is greater at the peripheral edge portion 26F of the haptic part 20 than the circumference of the haptic member 20F at the junction with the optical part 10F, so as to facilitate forward deformation. Each of these haptic members preferably subtends an angle of 90°, so that the gaps defined by the opposite lateral edges of the two haptic members also subtend an angle of 90°. With an embodiment of this kind, the surgeon is able, after implantation, to access the site via gaps 29F formed between the haptic members 20F to clean the posterior chamber beyond the implant.

The haptic part 20F of an embodiment of this kind is more flexible than the haptic part 20A of the first embodiment because it is divided into two haptic members 20F of reduced circumference. This increased flexibility, in the radial expansion zone 21F in particular, increases from the periphery of the haptic part towards the periphery of the optical part, thanks to the orientation of the lateral edges of the haptic members, whilst allowing good retention of the haptic part in the capsular sac thanks to the circumference of these haptic members at the level of the peripheral edge portion.

At least the major part of the lateral edges 29F of the haptic members 20F is substantially radial because, as shown here, the portion of the lateral edges corresponding to the junction zone of each haptic member 20F is slightly flared where it approaches the optical part 10F. Likewise, one or more of these lateral edges may be provided with a notch as a marker for checking that the implant is correctly oriented.

The overall diameter of an intraocular lens of the above kind is preferably slightly greater than the diameter of the capsular sac at the level of the equator.

In a variant of this embodiment that is not shown, the haptic part comprises three or even four haptic members of the same general shape as the haptic members of the embodiment shown in FIGS. 21 and 22, with the circumference of, and the gaps between, the haptic members reduced proportionately.

In a variant shown in FIG. 18 of the second embodiment shown in FIGS. 21 and 22, the haptic part 20 comprises two haptic members 20C extending in opposite directions from the peripheral edge portion 11C of the optical part 10C. Each haptic member 20C has the same radial section as the haptic part 20 of the embodiment shown in FIGS. 1 and 2. The corresponding parts are designated by the same reference numbers with the suffix C.

In a variant shown in FIG. 16 of the first embodiment shown in FIGS. 1 and 2, the haptic part 20 has a plurality of notches 27A disposed symmetrically about the optical axis A-A of the implant. The corresponding parts of the embodiment shown in FIGS. 1 and 2 are designated by the same reference numbers with the suffix A. The notches pass partly or completely through the annular undulations. The haptic part 20 is preferably provided with four notches 27A disposed at 90° to each other about the optical axis. Each of the notches 27A has a closed, rounded and preferably semicircular interior end 28A approximately 1 mm from the edge of the periphery 11A of the optical part 10 and opposite and parallel rectilinear edges 29A extending from the rounded end in a more or less radial direction and as far as the peripheral edge portion 26A of the haptic part 20.

In another variant shown in FIGS. 19 and 20 of the first embodiment shown in FIGS. 1 and 2, the haptic part 20 comprises a plurality of radial arms and three arms 20D each arm extending in the radial direction between the peripheral edge portion 11D of the optical part 10 and the peripheral edge portion 26D of the haptic part 20. Each of the radial arms 20D has the same radial section as the haptic part 20, the corresponding parts of the embodiment shown in FIGS. 1 and 2 having the same reference numbers with the suffix D. In this variant, the haptic arms 20D have a greater circumferential width at the junction with the peripheral edge portion 26D than at the junction with the peripheral edge portion of the optical part 11D. As shown here, these radial arms subtend an angle at the center of 60°. The gaps between the radial arms subtend the same angle at the center. The angle that the radial arms subtend at the center is preferably from 40° to 80°. Moreover, the lateral edges of the radial arms may be parallel to each other. In all cases, the width of each of the arms should be greater than or equal to 1 mm. In the embodiment shown in FIGS. 19 and 20 the surgeon is able, after implantation, to access the site through the closed contour gaps 29 formed between the radial arms 20D.

As shown diagrammatically in FIG. 7, in the first and second embodiments the length L1 of the haptic part 20 between the periphery 11 of the optical part and the peripheral edge portion 26 of the radial expansion zone 21 at rest is of the order of 2.5 mm to 3.0 mm and always significantly less than the length L2 of the haptic part 20 between the periphery 11 of the optical part and the peripheral edge portion 26 of the radial expansion zone 21 in the expanded state, which is of the order of 3 mm to 4 mm after reduction or elimination of the undulations. The same applies to the variants of these embodiments.

FIGS. 10 and 11 show an accommodative intraocular lens 1 conforming to the first embodiment shown in FIGS. 1 and 2 and to the second embodiment shown in FIGS. 21 and 22 and to variants thereof implanted in a capsular sac SC following ablation and phacoemulsification of the crystalline lens and cleaning of the site. It may be inserted through a small incision in the sclerotic/cornea if the optical part and the haptic part are made at least in part from flexible material, such as an acrylic or hydrophilic polyHEMA silicone. This kind of implant may be folded or rolled in order to pass it through a small incision before it is deployed in the posterior chamber of the aphakic eye. Any folding or injection device may be used, and in particular an injector. In the rest position for far vision, shown in FIG. 11, anterior and posterior sharp edges or square corners 27, 28 at the outside edge 20 of the peripheral edge portion 26 are in contact with the capsular sac. The square corners are intended to limit or inhibit proliferation of epithelial cells, in particular on the posterior capsule, responsible for opacification of the latter, known as a secondary cataract, and necessitating YAG laser surgery. In this position, the radial expansion zone 21 is normally prestressed, because the overall diameter of the implant is slightly greater than the diameter of the capsular sac SC at the level of the equator. As shown here, the center of the convex posterior face of the optical part 10 is in contact with and espouses the posterior capsule CP, which maximizes the transmission of vitreous pressure, which is applied immediately to the optical part during pseudoaccommodation of the eye.

For near vision, the combination of the vitreous pressure in the corresponding central region of the optical part concomitant with displacement of the apex of the ciliary muscle and the equator of the capsular sac, both radially towards the center and axially forwards, encourages the displacement of the optical part 10 towards the forward accommodation position, as shown in FIG. 11. Application of vitreous hyperpressure and displacement of the apex of the ciliary muscle stretch the radial expansion zone 21, and the undulations 23, 24 and 25 of the bellows 22 are therefore flattened or even eliminated when the optical part is in the maximum accommodation position. The radial expansion zone 21 therefore adopts a globally frustoconical shape. It goes without saying that if the vitreous hyperpressure were less than approximately 200 Pa, one or more partially flattened undulations would remain.

For far vision, the apex of the ciliary muscle and the equator have the reverse kinetics, and the vitreous hyperpressure falls to the rest vitreous pressure, thereby reducing the forces acting both on the peripheral edge portion 29 and on the optical part 10. The haptic part 20 therefore resumes its rest configuration, by virtue of the radial expansion zone returning to its initial position, as shown in FIG. 10. In the rest position the optical part is preferably slightly forward of, or where applicable in, a plane perpendicular to the optical axis passing through the middle of the area of contact of the peripheral edge portion of the haptic part with the capsular sac. The variants of this embodiment function in the same way as has just been described.

FIGS. 3 and 4 show a third embodiment of an accommodative intraocular lens. It comprises an optical part 30 and a haptic part 40. The optical part 30 shown has a biconvex shape but may have other shapes, as already indicated.

In the embodiment shown in FIGS. 3 and 4, the haptic part 40 has an expansion zone 41 comprising an annular bellows 42 having two annular undulations 43 and 44. The first undulation 43 is situated in the immediate vicinity of the periphery 31 of the optical part 30 and is open on the anterior side. The second undulation 44 extends circumferentially around the first undulation and is open on the posterior side. In the preferred form shown here, the bellows 42 is substantially sinusoidal in radial section. However, the second undulation is deeper and wider than the first undulation.

The depth of the first undulation is preferably from 0.40 mm to 0.70 mm and that of the second undulation is preferably from 0.6 mm to 1.0 mm. The first undulation 43 subtends an angle from 50° to 70° and the second undulation 44 subtends an angle from 500 to 700. The thickness of the haptic part 40 in the bellows shaped expansion zone is of the order of 0.15 mm and that of the haptic part in the peripheral zone is 0.3 mm. The haptic part 40 comprises an annular peripheral gutter 46. The thickness of the haptic part 40 in the zone comprising the peripheral gutter is substantially greater than that in the zone comprising the bellows 42 and this zone is therefore substantially more rigid than the radial expansion zone 41. The gutter 46 has a concave anterior surface 48 and a convex posterior surface 49 that are substantially concentric. The gutter subtends an angle at the center from 90° to 180°, and more particularly of the order of 150°. The peripheral gutter has a maximum width in the axial direction from 0.5 mm to 1.5 mm. The plane perpendicular to the optical axis A-A of the optical part 30 in the largest diameter of zone the gutter passes through the periphery 31 of the optical part 30 or is slightly offset forward of this periphery. Once implanted, the largest diameter zone of the gutter is aligned with the equator of the capsular sac SC.

In the third embodiment, shown in FIGS. 3 and 4, the haptic part 40 extends all around the optical part 30 and is annularly continuous.

As shown diagrammatically in FIG. 8, the length L3 of the haptic part 40 between the periphery 31 of the optical part and the peripheral edge portion 46 of the radial expansion zone 41 in the rest state is of the order of 2.4 mm to 2.8 mm and always substantially less than the length L4 of the haptic part 40 between the periphery 31 of the optical part and the peripheral edge portion 46 of the radial expansion zone 41 in the elongated state, which is of the order of 3 mm to 4 mm, after reduction or elimination of the undulations.

In a variant shown in FIG. 17 of this third embodiment, the posterior surface 47b of the gutter is provided with a plurality of bosses or protrusions 49b that are preferably of rounded shape. They cooperate with the capsular sac at the equator and are adapted to prevent the formation of or to reduce the size of transverse or radial creases between the periphery of the optical part 30 and the gutter of the haptic part 40 upon contraction of the periphery of the haptic part for near vision.

FIGS. 12 and 13 show the implant from FIGS. 3 and 4 implanted in a capsular sac SC, respectively in a rest position for far vision and in a maximum accommodation position. The choice of materials is the same as for the embodiment of FIGS. 1 and 2 and the implantation method is also the same.

In the rest position for far vision of the third embodiment, shown in FIG. 12, the convex posterior surface 47 of the gutter 46 is in contact with the capsular sac facing the equatorial zone of the zonules Z. The complementarity of this convex posterior surface and the corresponding part of the capsular sac constitutes a barrier to migration of epithelial cells towards the center of the posterior capsule CP and ensures good separation between the anterior and posterior leaves of the capsular sac, thereby restoring the fan-shaped termination of the zonule at the equator of the crystalline sac of aphakic eye.

This third embodiment of an accommodative intraocular lens functions in substantially the same manner as the first embodiment. Upon forward movement of the optical part for accommodation, the depth of the undulations decreases, or they even disappear, the haptic part progressively adopting a substantially frustoconical shape between the periphery of the optical part and the gutter.

FIGS. 5 and 6 show a fourth embodiment of an accommodative intraocular lens 3 which comprises an optical part 50 and a haptic part 60. The optical part 50 shown has a biconvex shape. Other shapes may be adopted.

In the embodiment of FIGS. 5 and 6, the haptic part 60 has an annular and substantially plane zone extending between the periphery 51 of the optical part 50 and a peripheral edge portion 66 with square corners 67, 68. In this embodiment, the radial expansion zone 61 is annular and consists at least in part of the annular zone between the periphery 51 of the optical part and the peripheral edge portion 66, and is made from a less rigid material and therefore has a higher elasticity. It is adapted to elongate on passing from the rest position to the accommodation position, and its inherent elasticity ensures that the optical part returns to the rest position when the vitreous hyperpressure and the position of the apex of the ciliary muscle revert to their initial values. In the rest position, the optical part is preferably slightly forward of, or where applicable in, a plane perpendicular to the optical axis and passing through the middle of the area of contact of the peripheral edge portion of the haptic part with the capsular sac.

In variants that are not shown of this fourth embodiment, the haptic part 60 may comprise two haptic members of the type shown in FIG. 18 or in FIGS. 21 and 22, disposed like those shown in FIG. 18.

In another variant that is not shown of this fourth embodiment, the plane annular zone is replaced partly or entirely by a bellows such as that shown in FIGS. 1 and 2 or in FIGS. 3 and 4. All or part of a bellows of this kind is therefore made from a material that is less rigid and therefore more elastic than the material of the peripheral edge portion, with the result that expansion is obtained in part through the reduction in the depth of, or the elimination of, the undulations and in part by the elongation of the zone formed of the material having a higher elasticity.

A two-material implant conforming to this fourth embodiment is preferably obtained by modifying the chemical and structural characteristics of the starting material, as described in published French patent application number 2.779.940, for example. It goes without saying that any other material or combination of materials may be used, on condition that the geometry and the functions of the implant of the present invention are retained. As shown diagrammatically in FIG. 9, in this embodiment the length L5 of the haptic part 60 between the periphery 51 of the optical part and the peripheral edge portion 66 of the radial expansion zone 61 in the rest state is of the order of 2.4 mm to 2.8 mm and always significantly less than the length L6 of the haptic part 60 between the periphery 51 of the optical part and the peripheral edge portion 66 of the radial expansion zone 61 in the elongated state, which is of the order of 3 mm to 4 mm.

FIGS. 14 and 15 show the implant of FIGS. 5 and 6 and its variants implanted in a capsular sac, respectively in the rest position for far vision and in the maximum accommodation position. The implantation method is the same as already described in relation to the first embodiment.

In the rest position for far vision represented in FIG. 15, the edge 66 is in contact through anterior and posterior square corners 67, 68 of the type already described with reference to the first embodiment and having the same functions.

This third embodiment of an accommodative intraocular lens functions in substantially the same way as described with reference to the other embodiments. Upon forward movement of the optical part for accommodation, the radial expansion zone is stretched, with the result that the distance between the periphery 51 of the optical part 50 and the peripheral edge portion 66 of the haptic part 60 is increased. If the radial expansion zone comprises one or more undulations, as in the variants of this embodiment, the latter are progressively reduced in depth and may even disappear when the optical part reaches its maximum accommodation position. In that position, the annular part 66 assumes a substantially frustoconical shape between the periphery of the optical part and the peripheral edge portion. The combination of the bellows and a material having a higher elasticity allows greater axial displacement for the purposes of accommodation.

Of course, the present invention is not limited to the embodiments described and shown, and encompasses any variant execution thereof. For example, the undulations in the haptic part are preferably sinusoidal, but other shapes may be suitable. Similarly, the thickness of the radial expansion zone may be uniform or vary. Likewise, when gaps and notches are provided about the axis of the optical part, their number and shape may vary. Finally, the optical part may comprise zones of rigid material and other zones of flexible material, allowing the optical part to be folded or rolled in order to insert it through a small incision. Configurations may be adopted for the peripheral zone of the haptic part other than a peripheral edge portion with a square corner or sharp edge and a peripheral edge portion consisting of a gutter.

1. Accommodative intraocular lens for implantation in the capsular sac, comprising a central optical part and a peripheral haptic part, the optical part having a forward accommodation position and a rest position for far vision, characterized in that the haptic part (20, 40, 60) comprises a radial expansion zone (21, 21A, 21B, 21C, 21D, 21F, 41, 61) allowing displacement of the optical part (10, 30, 50) towards the forward position.

2. Intraocular lens, characterized in that the radial expansion zone (21, 21A, 21B, 21C, 21D, 21F, 41) comprises a bellows (22, 22A, 22B, 22C, 22D, 22F, 42).

3. Intraocular lens according to claim 1, characterized in that the radial expansion zone (21, 21A, 21B, 21C, 21D, 21F, 41) comprises at least one undulation (23, 24, 25, 43, 44).

4. Intraocular lens according to claim 1, characterized in that the radial expansion zone (21, 21B, 41, 61) is substantially annular and extends circumferentially around the optical part.

5. Intraocular lens according to claim 1, characterized in that the haptic part (20C, 20F) comprises two symmetrical and diametrically opposite haptic members (21C, 21F).

6. Intraocular lens according to claim 5, characterized in that each haptic member has a circumference at the periphery of the haptic part greater than the circumference at the junction with the optical part (10).

7. Intraocular lens according to claim 5, characterized in that the haptic part comprises at least three circumferentially spaced haptic members.

8. Intraocular lens according to claim 5, characterized in that the gaps between the haptic members have the same circumference.

9. Intraocular lens according to claim 3 characterized in that the depth of the undulation(s) (23, 24, 25, 43, 44) is significantly reduced or eliminated in the forward position.

10. Intraocular lens according to claim 1, characterized in that it comprises a plurality of symmetrical radial notches (27A) open at the periphery of the haptic part (20) and in that the notches (27A) pass partly or totally through the annular undulation(s).

11. Intraocular lens according to claim 3, characterized in that there are at least two undulations of which one is open on the anterior side (23, 43) and the other or another is open on the posterior side (24, 44).

12. Intraocular lens according to claim 11, characterized in that the undulation (23, 43) open on the anterior side is formed at the periphery (11, 31) of the optical part (10, 30) and the undulation (24, 44) that is open on the posterior side extends around the undulation (23, 43) that is open on the anterior side.

13. Intraocular lens according to claim 12, characterized in that the undulation that is open on the anterior side is formed at the periphery of the optical part and the undulation that is open on the anterior side extends around the undulation that is open on the posterior side.

14. Intraocular lens according to claim 12, characterized in that the two undulations (23, 24, 43, 44) are substantially sinusoidal in radial section.

15. Intraocular lens according to claim 12, characterized in that, in the rest state of the lens, the bottom of the undulation (23) that is open on the anterior side is situated rearwardly of the periphery of the optical part (10).

16. Intraocular lens according to claim 12, characterized in that the bottom of the undulation that is open on the posterior side (44) is situated forwardly of the periphery of the optical part.

17. Intraocular lens according to claim 1, characterized in that the radial expansion zone (21, 41, 61) extends from the periphery of the optical part (10, 30, 50).

18. Intraocular lens according to claim 9, characterized in that each undulation (23, 24, 25, 43, 44) subtends an angle from 50° to 70°.

19. Intraocular lens according to claim 1, characterized in that the haptic part (20, 40, 60) comprises a peripheral edge portion (26, 26C, 26D, 26F, 66) with anterior square corners (27, 27C, 27F, 67) and posterior square corners (28, 28C, 28F, 68).

20. Intraocular lens according to claim 1, characterized in that the haptic part (20, 40, 60) is circumferentially continuous over the whole of its radial dimension.

21. Intraocular lens according to claim 1, characterized in that the radial expansion zone (61) is more flexible than the remainder of the haptic part (60).

22. Intraocular lens according to claim 21, characterized in that the radial expansion zone (61) comprises no undulations.

23. Intraocular lens according to claim 18, characterized in that the haptic part (40) comprises a peripheral gutter (46, 46B) whose maximum width in the axial direction is from 0.5 mm to 1.5 mm.

24. Intraocular lens according to claim 23, characterized in that the exterior surface of the peripheral gutter (46B) in its greatest diameter zone comprises protrusions or bosses (49B).

25. Intraocular lens according to claim 23, characterized in that the peripheral gutter (46) has a rounded external surface subtending an angle at the center from 90° to 180°.

26. Intraocular lens according to claim 1, characterized in that the haptic part (20) comprises a plurality of radial arms (20D) extending between the peripheral edge portion of the optical part (11D) and the peripheral edge portion of the haptic part (26D) and forming closed contour gaps (29D) between them.
